(19) United States
(12) Patent Application Publication
(10) Pub. No.: US 2010/0280094 A1
(43) Pub. Date: Nov. 4, 2010

(54) COMPOSITIONS AND METHODS TO TREAT MUSCULAR & CARDIOVASCULAR DISORDERS

(75) Inventors: Iwan Beuvink, Basel (CH); Jonathan Hall, Basel (CH); Jan Weiler, Basel (CH); Christian Schnell, Basel (CH); Mathias Mueller, Basel (CH); Martina Schinke-Braun, Cambridge, MA (US); Fabrizio Serluca, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/519,323

(22) PCT Filed: Dec. 13, 2007
(86) PCT No.: PCT/US2007/025535

§ 371 Date: Jun. 15, 2009

Related U.S. Application Data
(60) Provisional application No. 60/869,937, filed on Dec. 14, 2006.

(51) Int. Cl. A61K 31/7088; C07H 21/04; C07H 21/02; C12N 5/07; C12N 15/63; A61P 21/00; A61P 9/00

(52) U.S. Cl. 514/44 A; 536/23.1; 536/24.5; 435/325; 435/320.1

(57) ABSTRACT

The present invention relates to a novel microRNA, mir-208-2, implicated in muscular and cardiovascular disorders. The present invention also relates to oligonucleotide therapeutic agents (antisense oligonucleotides and/or double stranded oligonucleotides such as dsRNA) and their use in the treatment of muscular and cardiovascular disorders resulting from dysregulation of mir-208-2.

### FIELD OF THE INVENTION

This invention relates to a novel microRNA, mir-208-2, implicated in muscular and cardiovascular disorders. The present invention also relates to oligonucleotide therapeutic agents (antisense oligonucleotides and/or double stranded oligonucleotides) and their use in the treatment of muscular and cardiovascular disorders resulting from dysregulation of mir-208-2.

### BACKGROUND

MicroRNAs (miRNAs) are a class of non-coding RNA gene whose final product is a ˜22 nt functional RNA molecule. They are processed from endogenously encoded imperfect hairpin precursors as single-stranded RNAs. They appear to function via translational repression through base-pairing to the 3′-untranslated region (UTR) of target mRNAs (Griffith-Jones et al., 2006, Nucleic Acids Research, Vol. 34, D140-D144).

MicroRNA (miRNA) biogenesis is a complex, multi step process. Primary miRNA transcripts are transcribed by RNA polymerase II an can range in size from hundreds to thousands of nucleotides in length (pri-miRNA). MiRNAs can be traced back to two genomic sources. Some miRNAs are located within intronic regions of protein-coding genes. Others are located within the introns or exons of non-coding RNAs. Interestingly, pri-miRNAs can encode for a single miRNA but can also contain clusters of several miRNAs. The pri-miRNA is subsequently processed into a ˜70 nt hairpin (pre-miRNA) by the nuclear ribonuclease III (RNase III) endonuclease, Drosha. The pre-miRNA is than exported from the nucleus into the cytoplasm by Exporin5/RanGTP. In the cytoplasm, a second RNase III, Dicer, together with its dsRBD protein partner, cuts the pre-miRNA in the stem region of the hairpin thereby liberating an ˜21 nucleotide RNA-duplex. From the miRNA duplex, one strand enters the protein complex that repress target gene expression, the RNA-induced silencing complex (RISC), whereas the other strand is degraded. The choice of strand relies on the local thermodynamic stability of the miRNA duplex. The strand whose 5′ end is less stably paired is loaded into the RISC complex. The miRNAs loaded into the RISC complex appear to function via translational repression through base-pairing to the 3′-untranslated region (UTR) of target mRNAs Du, T. and Zamore, P. D. et al. micro-Primer: the biogenesis and function of microRNA. Development (2005) 132, 4645-4652. Currently 462 human miRNA sequences are deposited in miRBase (http://microrna.sanger.ac.uk) and it is suggested that this list will reach the 800 mark. The large numbers of miRNAs identified so far suggests that they might play complex roles in the regulation and fine tuning of biological processes. Indeed, several miRNAs have been implicated in cell proliferation control (mir-125b and let-7), hematopoietic B-cell lineage fate (mir-181), B-cell survival (mir-15a and mir-16-1), brain patterning (mir-430), pancreatic cell insulin secretion (mir-357), adipocyte development (mir-375) and muscle proliferation and differentiation (miR-1 and miR-133). Many miRNAs are located in genomic regions involved in cancer. For example, the cluster containing mir-16-1 and mir-15 is deleted and down-regulated in the majority of B-cell chronic lymphocytic leukemias (B-CLL; Calin, G. A., et al. MicroRNA-cancer connection: The beginning of a new tale. Cancer Res.(2006) 66, 7390-7394).

There exists a continuing unmet need for effective therapeutic treatment for the diseases and disorders that might be caused by dysregulated microRNA. This invention provides compounds that meet this need, and provide other benefits as well. The compounds of the invention are nucleic acids which can specifically target and treat dysregulated microRNA. One class is antisense DNA or RNA; the other class is double stranded RNA (dsRNA), which also includes a class known as short interfering RNA (siRNA).

siRNA are a novel class of therapeutic agent that have been shown to block gene expression in a highly conserved regulatory mechanism known as. RNA interference (RNAi). WO 99/32619 (Fire et al.) discloses the use of a dsRNA of at least 25 nucleotides in length to inhibit the expression of genes in C. elegans. siRNA has also been shown to degrade target RNA in other organisms, including plants (see, e.g., WO 99/53050, Waterhouse et al.; and WO 99/61631, Heifetz et al.), Drosophila (see, e.g., Yang, D., et al., Curr. Biol. (2000) 10:1191-1200), and mammals (see WO 00/44895, Limmer; and DE 101 00 586.5, Kreutzer et al.). This natural mechanism has now become the focus for the development of a new class of pharmaceutical agents for treating disorders that are caused by the aberrant or unwanted regulation of a gene.

Despite significant advances in the field of RNAi there remains a need for agents of diverse kinds that can treat diseases caused by novel molecular pathologies, such as dysregulated microRNAs.

### SUMMARY OF THE INVENTION

The present inventors have identified a new miRNA, mir-208-2, fulfilling the above needs. The present invention hence relates to an isolated nucleic acid molecule of less than 500 nucleotides characterized in that said isolated nucleic acid molecules comprise mir-208-2 (SEQ ID NO:7). In one embodiment, e.g. for targeting a pri-miRNA, the isolated nucleic acid molecule comprising mir-208-2 (SEQ ID NO:7) has a length of less than 200 nucleotides. In another embodiment, e.g. for targeting a pre-miRNA, the isolated nucleic acid molecule comprising mir-208-2 (SEQ ID NO:7) has a length of less than 80 nucleotides. Particular embodiments of the invention are isolated nucleic acid molecules selected from the group consisting of SEQ ID NO:1, SEQ ID NO:2, SEQ ID NO:3 and SEQ ID NO:4, or consisting of SEQ ID NO: 7.

The skilled person will immediately realize that the scope of the present invention also encompasses isolated nucleic acid molecules of less than 500 nucleotides consisting of a nucleic acid sequence which is complementary to ones described herein-above. For instance, an isolated nucleic acid consisting of SEQ ID NO:8.

Another embodiment of the invention is an isolated nucleic acid molecule having between 8 and 50 nucleotides in length and capable of hybridizing under physiological conditions, e.g. within a cell (cytoplasm or nucleus) or under conditions mimicking such conditions, to an isolated nucleic acid molecule as described herein-above, thus inhibiting the function of mir-208-2 (SEQ ID. NO:7), e.g. binding of mir-208-2 to its target.

Particular examples of such molecules are isolated nucleic acid molecules selected from the group consisting of SEQ ID NO:10 to SEQ ID NO:77.

It will be immediately evident to the person skilled in the art that the above isolated nucleic acid molecules can carry one or more chemical modifications e.g. selected from among a) a 3′ cap, b) a 5′ cap, c) a modified internucleoside linkage, or d) a modified sugar or base moiety.

A nucleic acid vector comprising a nucleic acid as described herein-above and at least one vector propagation sequence is also an embodiment of the invention. The present invention also relates to a nucleic acid vector comprising a nucleic acid as described herein-above and at least one vector propagation sequence

The isolated nucleic acid molecules of the invention, or nucleic acid vectors of the invention, can be used as a medicament, for instance in a lipid or polymer based therapeutic delivery system.

Such medicaments of the invention can be used for treating a muscular disorder in a subject having a muscular disorder.

A particular embodiment of such a muscular disorder is a cardiovascular disorder. Hence, the medicaments of the invention to treat a cardiovascular disorder in a subject, or for the preparation of a medicament for treating a muscular disorder or a cardiovascular disorder.

The present invention also encompasses a kit for use in diagnosing or determining a treatment strategy for a cardiovascular disorder. Typically, said kit will comprise a nucleic acid reagent comprising a nucleic acid molecule according to the present invention, in either RNA, DNA, mixed RNA or DNA, and optionally any chemical modifications.

The nucleic acid molecules according to the present invention can also be used for any other purposes, such as, for instance, experimental purposes. The present invention thus also includes any method of reducing or increasing expression of mir-208-2 wherein an isolated nucleic acid molecule according to the invention is used, for instance within in a cell. Similarly, the nucleic acid molecules according to the present invention can also be used as diagnostic probes or as experimental probes.

The present inventors moreover, realised that the expression of two other miRNAs, mir-208 and mir-499, closely correlate with the expression of mir-208-2. The present invention thus also relates to the use of an isolated nucleic acid molecule of less than 500 nucleotides characterized in that said isolated nucleic acid molecules comprise mir-208 (SEQ ID NO:6), and/or of an isolated nucleic acid molecule of less than 500 nucleotides characterized in that said isolated nucleic acid molecules comprise mir-499 (SEQ ID NO:9), and/or of an isolated nucleic acid molecule of less than 500 nucleotides comprising the complementary sequence of mir-208 (SEQ ID NO:6) or of mir-499 (SEQ ID NO:6) for the preparation of a medicament for treating a muscular disorder or a cardiovascular disorder, or for diagnosing a muscular disorder or a cardiovascular disorder.

### BRIEF DESCRIPTION OF THE DRAWINGS

FIG. 1. Alignment of the MYH6 intron harbouring human mir-208 with intron of MYH7 of zebra fish, human, chimp, dog and rat harbouring mir-208-2.

FIG. 2. RT-PCR of MYH6, MYH7 and 18S on RNA isolated from mouse hearts in different stages of development.

FIG. 3. Northern blot analysis of mir-208, mir-208-2, mir-499 and mir-206 on RNA isolated from mouse hearts in different stages of development. U6 snRNA was used as loading control. Lanes 11-14 correspond to 50 pg synthetic miRNAs.

### DETAILED DISCLOSURE OF THE INVENTION

This invention relates to the discovery of a novel microRNA, mir-208-2, implicated in muscular and cardiovascular disorders. It also relates to oligonucleotide therapeutic agents (antisense DNA/RNA and/or double stranded RNA) and their use in the treatment of muscular and cardiovascular disorders resulting from dysregulation of mir-208-2. Mir-208-2 is a gene located in intron 30 of the MYH7 gene. MYH7 (GenBank Accession NM_{—}000257: on chromosome 14). MYH7 is a motor contractile protein consisting of a globular head (contains actin and ATP binding sites) followed by a rod like tail sequence and is one of the building blocks constituting the thick myosin filaments. Each myosin filament contains two heavy chains and four light chains. The velocity of cardiac muscle contraction is controlled by the degree of ATPase activity in the head regions of the myosin molecules. The major determinant of myosin ATPase activity and, therefore, the speed of muscle contraction, depends on the relative amounts of the two myosin heavy chain isomers, MYH6 and MYH7. The MYH6 isoform, which exhibits high ATPase activity, has approximately four times more enzymatic activity than MYH7. Both MYH6 and MYH7 are expressed in different amounts in the human heart. In failing human hearts, MYH6 mRNA and protein levels are down regulated whereas MYH7 is upregulated

Transcription of Mir-208-2 is closely linked to MYH7 transcription, as it has no independent promoter. It is therefore transcribed only when transcripts of MYH7 are being generated. It is believed that the mir-208-2 microRNA is released from the MYH7 transcript when the pre-mRNA is processed and introns are removed. The residual intron sequence for intron 30 is then processed further to generate the full length mir-208-2.

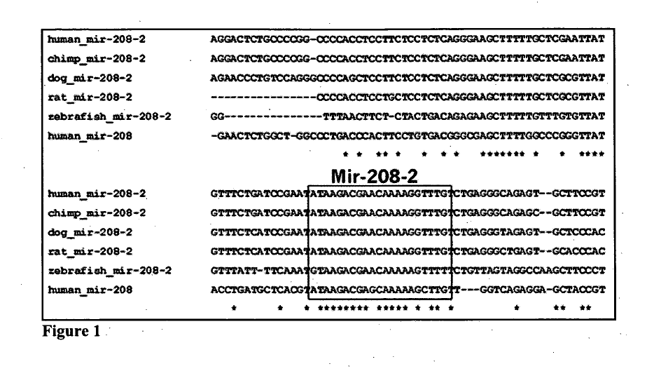

Mir-208-2 is highly conserved across vertebrates FIG. 1 illustrates an alignment of mir-208-2 between multiple species. The similarity with mir-208, a different microRNA residing in intron 28 of MYH6 is also illustrated.

Mir-208-2 genes identified by the inventors have the following sequences (including the flanking regions: the actual mir-208-2 sequence is in bold and underlined):

Homo sapiens
SEQ ID NO: 1
5′-CCCCACCTCCTTCTCCTCTCAGGGAAGCTTTTTGCTCGAATTAT
GTTTCTGATCCGAATATAAGACGAACAAAAGGTTTGTCTGAGGG-3′
Pan troglodytes
SEQ ID NO: 2
5′-CCCCACCTCCTTCTCCTCTCAGGGAAGCTTTTTGCTCGAATTAT
GTTTCTGATCCGAATATAAGACGAACAAAAGGTTTGTCTGAGGG-3′
Canis familiaris
SEQ ID NO: 3
5′-CCCCAGCTCCTTCTCCTCTCAGGGAAGCTTTTTGCTCGCGTTAT
GTTTCTCATCCGAATATAAGACGAACAAAAGGTTTGTCTGAGGG-3′
Rattus norvegicus
SEQ ID NO: 4
5′-CCCCACCTCCTGCTCCTCTCAGGGAAGCTTTTTGCTCGCGTTAT
GTTTCTCATCCGAATATAAGACGAACAAAAGGTTTGTCTGAGGG-3′
Danio rerio
SEQ ID NO: 5
5′-GTAAGACGAACAAAAAGTTTTT-3′

For sequence comparison, the previously known mir-208 from intron 28 of MYH6 is also provided.

| Homo sapiens mir-208 | |
|---|---|
| 5′-ATAAGACGAGCAAAAAGCTTGT-3′ | SEQ ID NO: 6 |

FIG. 1 illustrates the remarkable conservation of mir-208-2 between vertebrate species. From this alignment it is possible to identify the most highly conserved portion of this microRNA, which is concluded to be the functioning guide and anti-guide sequences as follows:

Guide Sequence (mir-208-2):

| | |
|---|---|
| 5′-ATAAGACGAACAAAAGGTTTGT-3′ | (SEQ ID NO: 7) |
| Anti-Guide Sequence | |
| 5′-ACAAACCTTTTGTTCGTCTTAT-3′ | (SEQ ID NO: 8) |

Unlike MYH6 and MYH7, MYH7B (GenBank Accession NM_{—}020884: on chromosome 20) is less well characterized. Based on its high degree of homology with MYH7, MYH7B is classified as a slow MYH isoform. To data, no clear function of MYH7B is described in the literature. In addition, no disease link is attributed to MYH7B dysfunction. Interestingly, like MYH6 and MYH7, MYH7B also harbors a miRNA within one of its introns namely mir-499.

| Homo sapiens mir-499 | |
|---|---|
| 5′-TTAAGACTTGCAGTGATGTTTAA-3′ | SEQ ID NO: 9 |

Bioinformatics analysis and the Examples included below indicate that the novel microRNA mir-208-2 is implicated in modulating signal transduction pathways involved in cardiac hypertrophy. An important utility of this microRNA is therefore as a target for the treatment of disorders and diseases that may be related to this.

The inventors disclose herein a variety of methods and compositions that have therapeutic utility. As a general overview, it is believed that reducing the level of the target microRNA provides therapeutic benefit in some cases. Such a decrease can be readily achieved by the use of antisense nucleic acid molecules, e.g. antisense DNA, directly binding to the target miRNA, e.g. mir-208-2. In other cases, increasing the level of the target microRNA, e.g. mir-208-2, will provide benefit. Such an increase can be readily obtained by the use of sense nucleic acid molecules and some dsRNA molecules, e.g. some siRNA, which will bind to the target of the miRNA, thus synergistically acting with said miRNA. The present invention is therefore directed to both types of therapeutic agent.

The following definitions are used throughout this specification and the claims.

An “isolated nucleic acid molecule” means that the material is removed from its original environment (e.g., the natural environment if it is naturally occurring). For example, a naturally-occurring polynucleotide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides could be part of a composition, and still be isolated in that such vector or composition is not part of its natural environment.

A “nucleic acid vector” is a nucleic acid sequence designed to be propagated and or transcribed upon exposure to a cellular environment, such as a cell lysate or a whole cell. A “gene therapy vector” refers to a nucleic acid vector that also carries functional aspects for transfection into whole cells; with the intent of increasing expression of one or more genes and/or proteins. In each case such vectors usually contain a “vector propagation sequence” which is commonly an origin of replication recognized by the cell to permit the propagation of the vector inside the cell. A wide range of nucleic acid vectors and gene therapy vectors are familiar to those skilled in the art.

As used herein, the phrases “therapeutically effective amount” and “prophylactically effective amount” refer to an amount that provides a therapeutic benefit in the treatment, prevention, or management of pathological processes mediated by dysregulation of mir-208-2. The specific amount that is therapeutically effective can be readily determined by ordinary medical practitioner, and may vary depending on factors known in the art, such as, e.g. the type of pathological processes, the patient's history and age, the stage of pathological processes, and the administration of other agents in combination.

As used herein, a “pharmaceutical composition” comprises a pharmacologically effective amount of a therapeutic agent of the invention and a pharmaceutically acceptable carrier. As used herein, “pharmacologically effective amount,” “therapeutically effective amount” or simply “effective amount” refers to that amount of an agent effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, the expression. “muscular disorder” includes, but is not limited to, cardiac pathology. This expression relates to any type of degenerative muscular disorder in which the primary pathology can be loss of striated muscle mass and/or function. This would include, but is not limited to, muscular dystrophies, trauma, and myasthenia gravis.

As used herein, a “transformed cell” is a cell into which a vector has been introduced from which a dsRNA molecule may be expressed. A cell comprising a nucleic acid which is supplied exogenously, such as the agents of this invention, whether transfected transiently or stably, is also considered a transformed cell.

Primary miRNA transcripts are transcribed by RNA polymerase II an can range in size from hundreds to thousands of nucleotides in length (pri-miRNA). pri-miRNAs can encode for a single miRNA but can also contain clusters of several miRNAs. The pri-miRNA is subsequently processed into a ˜70 nt hairpin (pre-miRNA) by the nuclear ribonuclease III (RNase III) endonuclease, Drosha. Thus, isolated nucleic acid molecules of the invention have various preferred length, dependending on their intended targets. When targeted to pri-miRNA a preferred length of about 500 nucleotides, e.g. 499, 450, 400, 350, 300, 250 nucleotides can be used. When targeted to pre-miRNA, preferred lengths vary between 100 and 200 nucleotides, e.g. 100, 120, 150, 180 or 200 nucleotides. In the cytoplasm, a second RNase III, Dicer, together with its dsRBD protein partner, cuts the pre-miRNA in the stem region of the hairpin thereby liberating an ˜21 nucleotide RNA-duplex. Thus isolated polynucleotides of e.g. 80, 70, 60, 50, 40, 30, 25, 21, 20, 19, 18 17, 16, 15, 14, 13, 12, 11, 10, 9 or 8 nucleotides in length are also considered in one embodiment of the invention.

The present inventors have discovered that the injection of an inhibitor of mir-208-2 into fertilized eggs of zebra fish (Dario rerio) lead to a drastic reduction of heart function (blood circulation, heart beatings, etc. . . . ). The “function of mir-208-2 (SEQ ID NO:7)” can hence be assessed with this assay or a similar assay one. Another possibility for assessing the “function of mir-208-2 (SEQ ID NO:7)” is the interaction of mir-208-2 with its targets, for instance its binding thereto.

In practicing the present invention, many conventional techniques in molecular biology, microbiology, and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring. Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

Therapeutic Agents

Certain of the therapeutic agents of the invention are described herein as siRNA comprising the anti-guide and guide sequences of mir-208-2 for use in increasing or decreasing the mir-208-2 activity in a cell. Other therapeutic agents of the invention are antisense DNA or RNA compositions which are useful to reducing the mir-208-2 activity in a cell.

dsRNA Therapeutics

The siRNA molecules according to the present invention mediate RNA interference (“RNAi”). The term “RNAi” is well known in the art and is commonly understood to mean the inhibition of one or more target genes in a cell by siRNA with a region which is complementary to the target gene. Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention. “siRNA” or “small-interfering ribonucleic acid” according to the invention has the meanings known in the art, including the following aspects; The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense DNA compounds referred to elsewhere in this specification. The other strand is known as the “anti-guide” sequence and because it contains the same sequence of nucleotides as the target sequence, it is known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

dsRNA, as used in this specification, comprises two fundamental classes. There is the siRNA, as described above. But also, where the two strands are part of one larger molecule, and therefore are connected by an uninterrupted chain of nucleotides between the 3′-end of one strand and the 5′end of the respective other strand forming the duplex structure, the connecting RNA chain is referred to as a “hairpin loop”, “short hairpin RNA” or “shRNA”. shRNA are normally transcribed from a nucleic acid vector and expressed in the target cell of interest.

The nucleic acid molecules of the invention can be any dsRNA that comprising SEQ ID NO: 7 or SEQ ID NO: 8 and will target the same cellular mRNA as mir-208-2 and/or mir-208-2 itself, as defined in the claims.

Guide Sequence

| | |
|---|---|
| 5′-ATAAGACGAACAAAAGGTTTGT-3′ | (SEQ ID NO: 7) |
| Anti-Guide Sequence | |
| 5′-ACAAACCTTTTGTTCGTCTTAT-3′ | (SEQ ID NO: 8) |

The nucleic acid molecules in accordance with the present invention comprise a region which is substantially identical to a region of the mRNA of the target gene. A region with 100% identity to the corresponding sequence of the target gene is suitable. This state is referred to as “fully complementary”. However, in view of the nature of miRNA and of their mechanism of action, the region may also contain one, two or three mismatches or more as compared to the corresponding region of the target gene, depending on the length of the region of the mRNA that is targeted, and as such may be not fully complementary. The most important feature is however that said molecules are able to specifically bind to mir-208-2 under physiological conditions, e.g. in a cell. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art, e.g. the method described in WO2005/059132. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of an siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted, into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918 arid will not be repeated here. Suitable modifications for delivery include chemical modifications selected from among:
a) a 3′ cap;
b) a 5′ cap,
c) a modified internucleoside linkage; or
d) a modified sugar or base moiety.

Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonuclotides with for instance phosphorothioates, chiral phosphorothioates or phosphorodithioates).

End modifications sometimes referred to herein as 3′ caps or 5′ caps may be of significance. Caps may consist of simply adding additional nucleotides, such as “T-T” which has been found to confer stability on an siRNA. Caps may consist of more complex chemistries which are known to those skilled in the art.

In an embodiment, the 3′ cap is a chemical moiety conjugated to the 3′ end via the 3′ carbon and is selected from among compounds of Formula I:

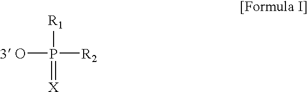

wherein

X is O or S

R1 and R2 are independently OH, NH2, SH, alkyl, aryl, alkyl-aryl, aryl-alkyl, where alkyl, aryl, alkyl-aryl, aryl-alkyl can be substituted by additional heteroatoms and functional groups, preferably a heteroatom selected from the group of N, O, or S or a functional group selected from the group OH, NH2, SH, carboxylic acid or ester;

or R1 and R2 may be of formula Y—Z where Y is O, N, S and Z is H, alkyl, aryl, alkyl-aryl, aryl-alkyl, where alkyl, aryl, alkyl-aryl, aryl-alkyl can be substituted by additional heteroatoms, preferably a heteroatom selected from the group of N, O, or S.

Examples of modifications on the sugar moiety include 2′ alkoxyribonucleotide, 2′ alkoxyalkoxy ribonucleotide, locked nucleic acid ribonucleotide (LNA), 2′-fluoro ribonucleotide, morpholino nucleotide.

The internucleoside linkage may also be modified. Examples of internucleoside linkage include phosphorothioate, phosphorodithioate, phosphoramidate, and amide linkages.

R1 may be OH;

R1 and R2 together may comprise from 1 to 24 C-atoms, from 1 to 12 C-atoms, from 2 to 10 C-atoms, from 1 to 8 or from 2 to 6 C-atoms. In another embodiment, R1 and R2 are independently OH, lower alkyl, lower aryl, lower alkyl-aryl, lower aryl-alkyl, where lower alkyl, lower aryl, lower alkyl-aryl; lower aryl-alkyl can be substituted by additional heteroatoms and functional groups as defined above. In another embodiment, R1 and R2 are not both OH.

The term “lower” in connection with organic radicals or compounds means a compound or radical which may be branched or unbranched with up to and including 7 carbon atoms, preferably 1-4 carbon atoms. Lower alkyl represents, for example, methyl, ethyl, n-propyl, isopropyl, n-butyl, sec-butyl, tert-butyl, n-pentyl and branched pentyl, n-hexyl and branched hexyl.

Examples of alkoxys include O-Met, O-Eth, O-prop, O-but, O-pent, O-hex.

Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918, both incorporated herein by reference. The reaction may be carried out in solution or, preferably, on solid phase or by using polymer supported reagents, followed by combining the synthesized RNA strands under conditions, wherein a siRNA molecule is formed, which is capable of mediating RNAi.

The present invention also encompasses an siRNA containing at least one modified nucleotide which is suitable for oral delivery. In functional terms this means siRNA will have suitable pharmacokinetics and biodistribution upon oral administration to achieve delivery to the target tissue of concern. In particular this requires serum stability, lack of immune response, and drug like behaviour. Many of these features of siRNA can be anticipated based on the standard gastric acid assays and standard serum assays disclosed elsewhere herein.

While the design of the specific therapeutic agent can take a variety of forms, certain functional characteristics will distinguish preferred dsRNA from other dsRNA. In particular, features such as good serum stability, high potency, lack of induced immune response, and good drug like behaviour, all measurable by those skilled in the art, will be tested to identify preferred dsRNA of the invention. In some situations, not all of these functional aspects will be present in the preferred dsRNA. But those skilled in the art are able to optimize these variables and others to select preferred compounds of the invention.

Any method can be used to administer a dsRNA of the present invention to a mammal containing dysregulated mir-208-2. For example, administration can be topical (e.g., vaginal, transdermal, etc); oral; or parenteral (e.g., by subcutaneous, intraventricular, intramuscular, or intraperitoneal injection, or by intravenous drip). Administration can be rapid (e.g., by injection), or can occur over a period of time (e.g., by slow infusion or administration of slow release formulations).

For example, dsRNAs formulated with or without liposomes can be topically applied directly to the tissue of interest. For topical administration, a dsRNA molecule can be formulated into compositions such as sterile and non-sterile aqueous solutions, non-aqueous solutions in common solvents such as alcohols, or solutions in liquid or solid oil bases. Such solutions also can contain buffers, diluents, and other suitable additives. Compositions for topical administration can be formulated in the form of transdermal patches, ointments, lotions, creams, gels, drops, suppositories, sprays, liquids, and powders. Gels and creams may be formulated using polymers and permeabilizers known in the art.

For parenteral, intrathecal, or intraventricular administration, a dsRNA molecule can be formulated into compositions such as sterile aqueous solutions, which also can contain buffers, diluents, and other suitable additives (e.g., penetration enhancers, carrier compounds, and other pharmaceutically acceptable carriers).

In addition, dsRNA molecules can be administered to a mammal using non-viral methods, such as biologic or abiologic means as described in, for example, U.S. Pat. No. 6,271,359. Abiologic delivery can be accomplished by a variety of methods including, without limitation, (1) loading liposomes with a dsRNA acid molecule provided herein; (2) complexing a dsRNA molecule with lipids or liposomes to form nucleic acid-lipid or nucleic acid-liposome complexes; or (3) providing a polymer based therapeutic delivery system. These techniques are generally well known in the art. A brief description follows.

A liposome can be composed of cationic and neutral lipids commonly used to transfect cells in vitro. Cationic lipids can complex (e.g., charge-associate) with negatively charged nucleic acids to form liposomes. Examples of cationic liposomes include, without limitation, lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin™. (Invitrogen/Life Technologies, Carlsbad, Calif.) and Effectene™. (Qiagen, Valencia, Calif.). In addition, systemic delivery methods can be optimized using commercially available cationic lipids such as DDAB or DOTAP, each of which can be mixed with a neutral lipid such as DOPE or cholesterol. In some cases, liposomes such as those described by Templeton et al. (Nature Biotechnology, 15: 647-652 (1997)) can be used. In other embodiments, polycations such as polyethyleneimine can be used to achieve delivery in vivo and ex vivo (Boletta et al., J. Am Soc. Nephrol. 7: 1728 (1996)). Additional information regarding the use of liposomes to deliver nucleic acids can be found in U.S. Pat. No. 6,271,359, PCT Publication WO 96/40964 and Morrissey, D. et al. 2005. Nat Biotechnol. 23(8):1002-7.

Biologic delivery can be accomplished by a variety of methods including, without limitation, the use of viral vectors. For example, viral vectors (e.g., adenovirus and herpesvirus vectors) can be used to deliver shRNA molecules to skin cells and cervical cells. Standard molecular biology techniques can be used to introduce one or more of the shRNAs provided herein into one of the many different viral vectors previously developed to deliver nucleic acid to cells. These resulting viral vectors can be used to deliver the one or more dsRNAs to cells by, for example, infection.

dsRNAs of the present invention can be formulated in a pharmaceutically acceptable carrier or diluent. A “pharmaceutically acceptable carrier” (also referred to herein as an “excipient”) is a pharmaceutically acceptable solvent, suspending agent, or any other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

In addition, dsRNA of the invention can be formulated into compositions containing the dsRNA admixed, encapsulated, conjugated, or otherwise associated with other molecules, molecular structures, or mixtures of nucleic acids. For example, a composition containing one or more dsRNA agents of the invention can also be combined with other therapeutic agents used in the treatment of similar disorders.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in formulation a range of dosage for use in humans. The dosage of compositions of the invention lies generally within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. In any event, the administering physician can adjust the amount and timing of dsRNA administration on the basis of results observed using standard measures of efficacy known in the art or described herein.

Antisense DNA Therapeutic Agents

The antisense oligonucleotides (herein sometimes called “antisense”) of the invention are designed to target mir-208-2 and reduce the level of its transcript. As such, the antisense may target any part of this mir-208-2 to knock down its level in a cell.

Antisense compounds are commonly used as research reagents and diagnostics, and for many years have been the subject of therapeutic investigation. Antisense oligonucleotides are able to inhibit gene expression with exquisite specificity and are often used by those of ordinary skill to elucidate the function of particular genes. Antisense compounds are also used, for example, to distinguish between functions of various members of a biological pathway.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term “antisense” refers to an oligomer or polymer of deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted antisense are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention contemplates other oligomeric antisense compounds, including but not limited to oligonucleotide mimetics such as are described below. The antisense compounds in accordance with this invention preferably comprise from about 8 to about 30 nucleobases. Particularly preferred are antisense oligonucleotides comprising from about 8 to about 30 nucleobases (i.e. from about 8 to about 30 linked nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkyiphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. Techniques for the synthesis of antisense compounds containing oligonucleotides with modified backbones or non-natural internucleoside linkages as described above may be achieved using conventional methodologies, and are familiar to one of skill in the art. Representative United States patents that teach the preparation of the above phosphorus-containing linkages include, but are not limited to, U.S. Pat. Nos. 3,687,808; 4,469,863 and 5,625,050; each of which is incorporated by reference herein in its entirety.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Synthesis of such oligonucleotides may be achieved by one of skill in the art according to conventional methods, for example, as described in U.S. Pat. Nos. 5,034,506; 5,166,315 or 5,677,439 each of which is incorporated by reference herein in its entirety.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is, referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides having morpholino backbone structures as described in U.S. Pat. No. 5,034,506. Also preferred are oligonucleotides with phosphorothioate backbones and oligonucleosides with heteroatom backbones, and in particular —CH2-NH—O—CH2-, —CH2-N(CH3)-O—CH2- [known as a methylene(methylimino) or MMI backbone], —CH2-O—N(CH3)-CH2-, —CH2-N(CH3)-N(CH3)-CH2- and —O—N(CH3)-CH2-CH2- [wherein the native phosphodiester backbone is represented as —O—P—O—CH2-] as described in U.S. Pat. No. 5,489,677, and the amide backbones as described in U.S. Pat. NO: 5,602,240.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH2)n O]m CH3, O(CH2)n OCH3, O(CH2)n NH2, O(CH2)n CH3, O(CH2)n ONH2, and O(CH2)n ON[(CH2)n CH3)]2, where n and m are from 1 to about 10. Other preferred oligonucleotides comprise one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2 CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH2 CH2 OCH3, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH2)2 ON(CH3)2 group, also known as 2′-DMAOE. A further preferred modification of this category is the bicyclic class of modifications known collectively as LNAs (Locked Nucleic Acids) as described in Rajwanshi et al., Angew. Chem. Int. Ed. 2000, 39, 1656-1659.

Other preferred modifications include 2′-methoxy (2′-O—CH3), 2′-amninopropoxy (2′-OCH2 CH2 CH2 NH2) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. One of skill in the art may use conventional methods to created such modified sugar structures. Representative United States patents that teach the preparation of such modified sugar structures include, but are not limited to, U.S. Pat. Nos. 4,981,957; 5,118,800 and 5,700,920 each of which is incorporated by reference herein in its entirety.

Antisense oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

One of skill in the art is able, to prepare modified nucleobases according to methods that are well known in the art. For example, representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302 and 5,134,066 each of which is incorporated by reference herein in its entirety.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991; 10, 1111-1118; Kabanov et al., FEBS Lett., 1990, 259, 327-330; Svinarchuk.et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Representative United States patents that teach the preparation of such oligonucleotide conjugates include, but are not limited to, U.S. Pat. Nos. 4,828,979; 4,948,882 and 5,688,941 each of which is incorporated by reference herein in its entirety.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. One of skill in the art may prepare these hybrid structures according to conventional methods. Representative United States patents that teach the preparation of such hybrid structures include, but are not limited to, U.S. Pat. Nos. 5,013,830; 5,149,797 and 5,700,922, and each of which is incorporated by reference herein in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

In addition, the skilled person will immediately understand that the antisense molecules of the invention do not have to target mir-208-2 per se, but can also target the mRNA comprising mir-208-2, for instance the pri-miRNA or the pre-miRNA.

Table 1 sets out preferred antisense sequences for down regulating mir-208-2. These sequences can be employed with any of the chemical modifications disclosed herein.

TABLE 1

| Antisense sequence | SEQ ID NO: |
|---|---|
| 5′-CCCTCAGACAAACCTTTTGTT-3′ | 10 |
| 5′-CCTCAGACAAACCTTTTGTTC-3′ | 11 |
| 5′-CTCAGACAAACCTTTTGTTCG-3′ | 12 |
| 5′-TCAGACAAACCTTTTGTTCGT-3′ | 13 |
| 5′-CAGACAAACCTTTTGTTCGTC-3′ | 14 |
| 5′-AGACAAACCTTTTGTTCGTCT-3′ | 15 |
| 5′-GACAAACCTTTTGTTCGTCTT-3′ | 16 |
| 5′-ACAAACCTTTTGTTCGTCTTA-3′ | 17 |
| 5′-CAAACCTTTTGTTCGTCTTAT-3′ | 18 |
| 5′-AAACCTTTTGTTCGTCTTATA-3′ | 19 |
| 5′-AACCTTTTGTTCGTCTTATAT-3′ | 20 |
| 5′-ACCTTTTGTTCGTCTTATATT-3′ | 21 |
| 5′-CCTTTTGTTCGTCTTATATTC-3′ | 22 |
| 5′-CTTTTGTTCGTCTTATATTCG-3′ | 23 |
| 5'-TTTTGTTCGTCTTATATTCGG-3' | 24 |
| 5′-TTTGTTCGTCTTATATTCGGA-3′ | 25 |
| 5′-TTGTTCGTCTTATATTCGGAT-3′ | 26 |
| 5′-TGTTCGTCTTATATTCGGATC-3′ | 27 |
| 5′-GTTCGTCTTATATTCGGATCA-3′ | 28 |
| 5′-TTCGTCTTATATTCGGATCAG-3′ | 29 |
| 5′-TCGTCTTATATTCGGATCAGA-3′ | 30 |
| 5′-CGTCTTATATTCGGATCAGAA-3′ | 31 |
| 5′-GTCTTATATTCGGATCAGAAA-3′ | 32 |
| 5′-TCTTATATTCGGATCAGAAAC-3′ | 33 |
| 5′-CTTATATTCGGATCAGAAACA-3′ | 34 |
| 5′-TTATATTCGGATCAGAAACAT-3′ | 35 |
| 5′-TATATTCGGATCAGAAACATA-3′ | 36 |
| 5′-ATATTCGGATCAGAAACATAA-3′ | 37 |
| 5′-TATTCGGATCAGAAACATAAT-3′ | 38 |
| 5′-ATTCGGATCAGAAACATAATT-3′ | 39 |
| 5′-TTCGGATCAGAAACATAATTC-3′ | 40 |
| 5′-TCGGATCAGAAACATAATTCG-3′ | 41 |
| 5′-CGGATCAGAAACATAATTCGA-3′ | 42 |
| 5′-GGATCAGAAACATAATTCGAG-3′ | 43 |
| 5′-GATCAGAAACATAATTCGAGC-3′ | 44 |
| 5′-ATCAGAAACATAATTCGAGCA-3′ | 45 |
| 5′-TCAGAAACATAATTCGAGCAA-3′ | 46 |
| 5′-CAGAAACATAATTCGAGCAAA-3′ | 47 |
| 5′-AGAAACATAATTCGAGCAAAA-3′ | 48 |
| 5′-GAAACATAATTCGAGCAAAAA-3′ | 49 |
| 5′-AAACATAATTCGAGCAAAAAG-3′ | 50 |
| 5′-AACATAATTCGAGCAAAAAGC-3′ | 51 |
| 5′-ACATAATTCGAGCAAAAAGCT-3′ | 52 |
| 5′-CATAATTCGAGCAAAAAGCTT-3′ | 53 |
| 5′-ATAATTCGAGCAAAAAGCTTC-3′ | 54 |
| 5′-TAATTCGAGCAAAAAGCTTCC-3′ | 55 |
| 5′-AATTCGAGCAAAAAGCTTCCC-3′ | 56 |
| 5′-ATTCGAGCAAAAAGCTTCCCT-3′ | 57 |
| 5′-TTCGAGCAAAAAGCTTCCCTG-3′ | 58 |
| 5′-TCGAGCAAAAAGCTTCCCTGA-3′ | 59 |
| 5′-CGAGCAAAAAGCTTCCCTGAG-3′ | 60 |
| 5′-GAGCAAAAAGCTTCCCTGAGA-3′ | 61 |
| 5′-AGCAAAAAGCTTCCCTGAGAG-3′ | 62 |
| 5′-GCAAAAAGCTTCCCTGAGAGG-3′ | 63 |
| 5′-CAAAAAGCTTCCCTGAGAGGA-3′ | 64 |
| 5′-AAAAAGCTTCCCTGAGAGGAG-3′ | 65 |
| 5′-AAAAGCTTCCCTGAGAGGAGA-3′ | 66 |
| 5′-AAAGCTTCCCTGAGAGGAGAA-3′ | 67 |
| 5′-AAGCTTCCCTGAGAGGAGAAG-3′ | 68 |
| 5′-AGCTTCCCTGAGAGGAGAAGG-3′ | 69 |
| 5′-GCTTCCCTGAGAGGAGAAGGA-3′ | 70 |
| 5′-CTTCCCTGAGAGGAGAAGGAG-3′ | 71 |
| 5′-TTCCCTGAGAGGAGAAGGAGG-3′ | 72 |
| 5′-TCCCTGAGAGGAGAAGGAGGT-3′ | 73 |
| 5′-CCCTGAGAGGAGAAGGAGGTG-3′ | 74 |
| 5′-CCTGAGAGGAGAAGGAGGTGG-3′ | 75 |
| 5′-CTGAGAGGAGAAGGAGGTGGG-3′ | 76 |
| 5′-TGAGAGGAGAAGGAGGTGGGG-3′ | 77 |

The antisense compounds of the invention are synthesized in vitro but may include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption and conventional methods for so doing exist and are familiar to one of skill in the art. For example, representative United States patents include, but are not limited to, U.S. Pat. Nos. 5,108,921; 5,354,844 and 5,595,756, each of which is incorporated by reference herein in its entirety.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. The term “pharmaceutically acceptable salts” refers to physiologically and pharmaceutically acceptable salts of the compounds of the invention: i.e., salts that retain the desired biological activity of the parent compound and do not impart undesired toxicological effects thereto. Such compounds may be prepared according to conventional methods by one of skill in the art. (Berge et al., “Pharmaceutical Salts,” J. of Pharma Sci., 1977, 66, 1-19).

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 to Imbach et al.

The antisense compounds of the present invention can also be utilized for diagnostics, therapeutics, prophylaxis and as research reagents and kits. For therapeutics, an animal, preferably a human, suspected of having a disease or disorder which can be treated by modulating the expression of mir-208-2 is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding mir-208-2, enabling sandwich and other assays to easily be constructed to exploit this fact. Hybridization of the antisense oligonucleotides of the invention with a nucleic acid encoding mir-208-2 can be detected by means known in the art. Such means may include conjugation of an enzyme to the oligonucleotide, radiolabelling of the oligonucleotide or any other suitable detection means. Kits using such detection means for detecting the level of mir-208-2 in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Use of Compositions of the Invention

As described above, the compositions of the invention find use in multiple settings, including but not limited to research, diagnostics, and therapeutics.

For therapeutic use, the compositions described herein can be used to treat diseases and conditions caused by dysregulation of mir-208-2. As set forth herein this novel microRNA is found expressed primarily in muscle tissue, particularly cardiac tissue, and is associated with MYH7 transcription and expression. The diseases associated with dysregulation of mir-208-2 include but are not limited to muscle disorders and cardiac disorder; and may include such diseases correlated with mutations in MYH6, MYH7 or MYH7B. Dysregulated expression of miRNAs could be the cause of the progression of the disease and would therefore qualify them as potential therapeutic targets either by inhibition of miRNAs or reintroduction of dsRNA using siRNA or shRNA with a suitable delivery systems.

For example, compounds of the invention can be used to treat Atrial fibrillation (AF), the most common sustained arrhythmia and is associated with extensive structural, contractile and electrophysiological remodeling with the aim to stabilize AF in the long run (Allessie, M., J. Ausma, and U. Schotten, Electrical, contractile and structural remodeling during atrial fibrillation. Cardiovasc Res, 2002. 54(2): p. 230-46. AF is associated with increased expression of ventricular myosin isoforms in atrial myocardium and is regarded as part of a dedifferentiation process. Interestingly, in AF myocardium, functional classes of genes that are characteristic of ventricular myocardium were found to be up-regulated whereas functional classes predominantly expressed in atrial myocardium were down-regulated. (Barth, A. S., et al., Reprogramming of the Human Atrial Transcriptome in Permanent Atrial Fibrillation: Expression of a Ventricular-Like Genomic Signature 10.1161/01.RES.0000165480.82737.33. Circ Res, 2005, 96(9): p. 1022-1029). One of the genes found to be up-regulated in AF was MYH7B, the gene harboring mir-499 in one of its introns. The isoenzyme shift of the MYH family members observed in human atrial tissue is thought to be an early adaptation to hemodynamic overload (Buttrick, P. M., et al., Myosin isoenzyme distribution in overloaded human atrial tissue. Circulation, 1986. 74(3): p. 477-83; Yazaki, Y., et al., Molecular adaptation to pressure overload in human and rat hearts. J Mol Cell Cardiol, 1989. 21 Suppl 5: p. 91-101.).

In another example, compounds find use in Hypertrophic cardiomyopathy (HCM) which has been characterized by a small, markedly hypertrophied, hypercontractile left ventricle (LV) (Maron, B. J., Hypertrophic cardiomyopathy: a systematic review. Jama, 2002. 287(10): p. 1308-20). Human ventricular muscle express both MYH6 and MYH7 with MYH7 being predominant. No marked differences in MYH7 expression are found during hypertrophy. However, overloaded human ventricular-muscle appears to lose the small amount of MYH6 it normally contains, since this form is not detected either in autopsy material of patients suffering from hypertensive disease or in perioperative biopsies of patients with valvular heart disease (Schwartz, K., et al., Left ventricular isomyosins in normal and hypertrophied rat and, human hearts. Eur Heart J, 1984. 5 Suppl F: p. 77-83. Mercadier, J. J., et al., Myosin isoenzymes in normal and hypertrophied human, ventricular myocardium. Circ Res, 1983. 53(1): p. 52-62).

The microRNAs identified, mir-208-2, mir-208 and mir-499, could also serve as biomarkers in the detection of early onset of atrial fibrillation or for hypertrophic cardiomyopathy.

Pharmaceutical compositions and formulations of the compounds of the invention for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids, according to conventional methods, by one of skill in the art.

The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.). Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical compositions suitable for parenteral administration may be formulated in aqueous solutions, preferably in physiologically compatible buffers such as Hanks' solution, Ringer's solution; or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art. The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical:composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those-skilled in the art. For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Alternative uses for the compositions of the invention include non-therapeutic uses including but not limited to biomarker indications, diagnostics, and research use. Those skilled in the art can now make use of the compounds of the invention for these purposes based on the novel discovery of mir-208-2 disclosed herein. Methods of research use of particular interest include reducing or increasing expression in a cell of the mir-208-2 microRNA. As such the invention also contemplates a kit for use in diagnosing or determining a treatment strategy for a cardiovascular or muscle disorder, a method of reducing or increasing expression in a cell of mir-208-2, mir-208 and/or mir-499, and a method of reducing or increasing mir-208-2, mir-208 and/or mir-409 activity in a cell

### Examples

Identification and Characterization of miRNA-208-2.

MicroRNA expression profiling was achieved according to the following protocol:

RNA Isolation

Rats and mice were thoracectomized after having been deeply anesthetized with isoflurane (3%, 20 L/min) and perfused through the left ventricle of the heart. The left ventricle was punctured with a 23 gauge needle from a winged infusion set (SV-19BLK; Termudo, Elkton, Md.), which was connected to an airtight pressurized syringe containing the rinsing solution (NaCl 0.9% with 250,000 U/l heparin at 38° C.). The right atrium was punctured to provide outflow, and the perfusate was infused under a precise controlled pressure of 120 mm Hg. The perfusion was continued for 2 min at a constant rate (20 ml). Organs were isolated, snap frozen in liquid Nitrogen and stored at −80° C. The organs were homogenized in the presence of 1 ml Trizol® Reagent (Life-Technologies™, cat no: 15596-018)Trizol per 100 mg tissue using a polytron homogenizer according to the protocols provided by the manufacturer. RNA was dissolved in RNAse-free water and stored at −80° C.

Northern DNA Probes

Probes against mir-208 (5′-acaagctttttgctcgtcttat-3′), mir-208-2 (5′-acaaaccttttgttcgtcttat-3′), mir-499 (5′-aaacatcactgcaagtctt-3′), mir-206 (5′-ccacacacttccttacattcca-3′) and U6 snRNA (5′-gccatgctaatcttctctgtatc-3′) were 5′-digoxigenin-labeled. All probes and synthetic miRNA sequences were obtained from Microsynth GmbH.

Northern Blotting

Northern blot analysis was performed using digoxigenin-labeled DNA oligonticleotides. In brief, 5 μg of total RNA from each tissue were separated on denaturing 15% polyacrylamide/7M urea gels (Invitrogen, cat no: EC68855BOX) run in 1×TBE. Resolved RNA was transferred for 90 min at 0.8 mA/cm² in 0.5×TBE to positively charged Nylon membrane (Roche, cat no: 1209299).After UV-cross-linking at 120 mJ, the membranes were washed with 2×SSC and blocked for 20 minutes with DIG Easy Hyb-buffer (Roche, cat no: 11603558001). After blocking the membranes were incubated for 60 min with DIG Easy Hyb-buffer containing 1 pmol/ml 5′-DIG labelled DNA oligo. The membranes were rinsed with 0.1%SDS/2×SSC followed by two washes 2×SSC. Membranes were subsequently washed, blocked (Roche, cat no: 1585762, Roche) and incubated with an alkaline phosphatase conjugated anti-digoxigenin antibody (Roche, cat no: 1093274) according to the manufacturers protocol. Membranes were incubated in ready-to-use CDP-Star (Roche, cat no: 2041677) and chemiluminescense was detected using the ChemiDoc XRS (BioRad). Results: miRNA expression profiling of different mouse tissue revealed that the expression both mir-208 and mir-499 is highly enriched in the heart. Northern blot analysis of both mouse and rat tissues confirms the observed expression pattern of these miRNAs (data not shown).

In more detail, mir-208 was found to be highly expressed in atrium and ventricle of the heart and the expression is conserved in both mouse and rat. Mir-499 on the other hand is restricted to the ventricle regions of the heart. Mir-206 is mainly expressed in muscle with low level expression detected in the heart. With these findings we identify that the expression of mir-499 (Bentwich, I., et al., Identification of hundreds of conserved and nonconserved human microRNAs. Nat Genet, 2005.) is highly enriched in ventricle regions of the heart; and confirm the heart and muscle enriched expression profiles of mir-208 (Lagos-Quintana, M., et al., Identification of tissue-specific microRNAs from mouse. Curr Biol, 2002. 12(9): p. 735-9.) and mir-206 (Sempere, L. F., et al., Expression profiling of mammalian microRNAs uncovers a subset of brain-expressed microRNAs with possible roles in murine and human neuronal differentiation. Genome Biol, 2004. 5(3): p. R13.).

Mir-208, previously cloned from heart (Lagos-Quintana, M., et al., New microRNAs from mouse and human. Rna, 2003. 9(2): p. 175-9.) is located within an intron of the myosin heavy chain 6 gene and is highly conserved amongst mammals. Interestingly, mir-499 (Bentwich, et al. supra) is located within an intron of the human myosin heavy chain 7B gene. The miRNA mir-499 is highly conserved amongst an even larger set of species compared to mir-208 (Zebrafish, human, chimp, dog, rat, mouse and Xenopus).

This data suggests that both gene and miRNA functions are conserved and it is therefore surprising that a similar level of conservation of mir-499 is not found in mir-208. Interestingly, in zebrafish (Danio rerio) ventricle myosin heavy chain (vmhc) is a closely related homologue of the human MYH6 and its transcript has a similar intron/exon structure.

Alignment of the MYH6 intron that harbors the mir-208 sequence with the vmhc transcript, revealed the presence of a similar mir-208 sequence with 4 nucleotides that differ (FIG. 1). Interestingly, vmhc is more related to the mammalian MYH7 family member rather than the MYH6 member since both vmhc and MYH7 have a slow contractile velocity due to a low rate of ATP hydrolysis. MYH6 on the other hand belongs to the ‘fast’-isoform (Weiss, A. and L. A. Leinwand, The mammalian myosin heavy chain gene family. Annu Rev Cell Dev Biol, 1996. 12: p. 417-39). Surprisingly, alignment of the vmhc intron harboring mir-208-2 with the mammalian MYH7 introns revealed a novel mir-208-like miRNA, herein now called mir-208-2.

In summary, we have identified novel miRNA sequences embedded within an intron of the zebrafish gene vmhc and the mammalian MYH7 gene.

### Example

### Characterization of mir-208-2

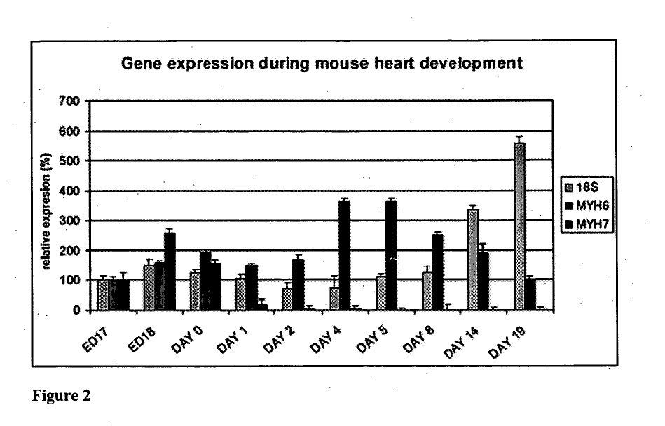

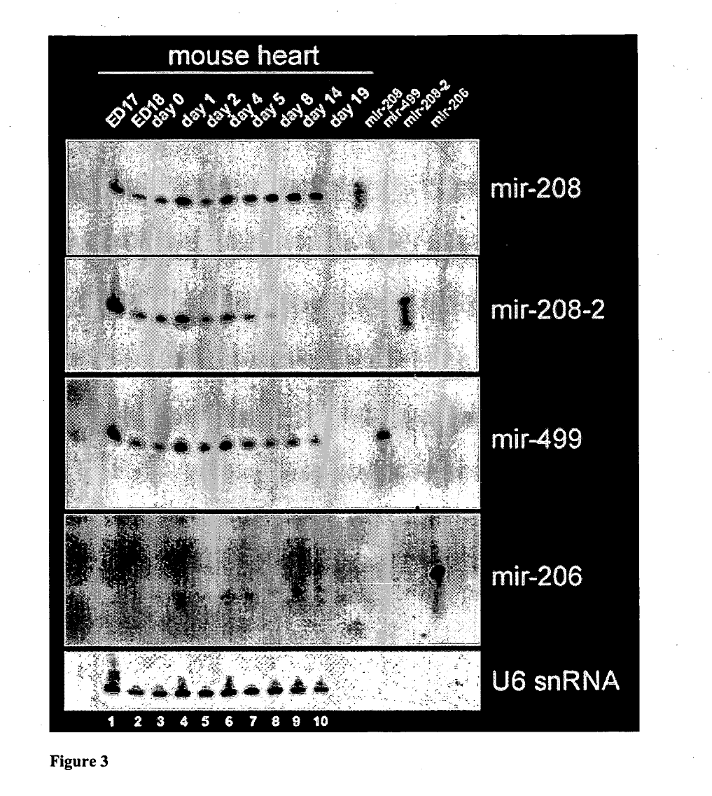

In normal mouse and rat hearts, MYH7 is only expressed during neonatal development of the heart (Lyons, G. E., et al., Developmental regulation of myosin gene expression in mouse cardiac muscle. J Cell Biol, 1990. 111(6 Pt 1): p. 2427-36.). However, MYH7 and other fetal genes are re-expressed when the heart is exposed to pressure overload resulting in hypertrophy. In order to verify the existence of mir-208-2, total RNA was isolated from mouse hearts during different stages of development. RT-PCR (FIG. 2) and Northern blot analysis (FIG. 3) was performed to confirm gene expression and miRNA expression respectively.

RNA Isolation

Mouse hearts of different developmental stages ranging from embryonic day (ED)17 to 19 days after birth were isolated, snap frozen in liquid Nitrogen and stored at −80° C. The organs were homogenized in the presence of 1 ml Trizol® Reagent (Life-Technologies™, cat no: 15596-018)Trizol per 100 mg tissue using a polytron homogenizer according to the protocols provided by the manufacturer. RNA was dissolved in RNAse-free water and stored at −80° C.

Northern DNA Probes

Probes against mir-208 (5′-acaagctttttgctcgtcttat-3′), mir-208-2 (5′-acaaaccttttgttcgtcttat-3′), mir-499 (5′-aaacatcactgcaagtctt-3′), mir-206 (5′-ccacacacttccttacattcca-3′) and U6 snRNA (5′-gccatgctaatcttctctgtatc-3′) were 5′-digoxigenin-labeled. All probes and synthetic miRNA sequences were obtained from Microsynth GmbH.

Northern Blotting

Northern blot analysis was performed using digoxigenin-labeled DNA oligonucleotides. In brief, 5 μg of total RNA from each tissue were separated on denaturing 15% polyacrylamide/7M urea gels (Invitrogen, cat no: EC68855BOX) run in 1×TBE. Resolved RNA was transferred for 90 min at 0.8 mA/cm² in 0.5×TBE to positively charged Nylon membrane (Roche, cat no: 1209299).After UV-cross-linking at 120 mJ, the membranes were washed with 2×SSC and blocked for 20 minutes with DIG Easy Hyb-buffer (Roche, cat no: 11603558001). After blocking the membranes were incubated for 60 min with DIG Easy Hyb-buffer containing 1 pmol/ml 5′-DIG labelled complementary DNA oligo. The membranes were rinsed with 0.1%SDS/2×SSC followed by two washes 2×SSC. Membranes were subsequently washed, blocked (Roche, cat no: 1585762, Roche) and incubated with an alkaline phosphatase conjugated anti-digoxigenin antibody (Roche, cat no: 1093274) according to the manufacturers protocol. Membranes were incubated in ready-to-use CDP-Star (Roche, cat no: 2041677) and chemiluminescense was detected using the ChemiDoc XRS (BioRad).

Gene Expression Analysis

PCR-primer sets for MYH6 (Mm00440354_m1), MYH7 (Mm00600555_m1) and 18S (Hs99999901-s1) were obtained from Applied Biosystems (AB) using the one-step RT-PCR Master Mix reagents (AB, cat no: 4309169) according to the protocol provided by the manufacturer. All samples were measured in triplicate using the 7500 FAST Real-Time PCR System (AB).

We confirmed that MYH7 expression is restricted to the neonatal stages of mouse heart development . Two days after birth, MYH7 is no longer detected by RT-PCR. Mir-208-2 is expressed before birth and shortly after birth (day 8) but is virtually undetectable by day 14. Northern blot analysis of mir-208 correlates with the expression of MYH6 and is present during neonatal and post-natal stages.

Mir-208-2 expression in adult human heart: Unlike mouse and rat, the healthy human heart expresses both MYH6 and MYH7. Schiaffino et al. (Schiaffino, S., et al., Myosin changes in hypertrophied human atrial and ventricular myocardium. A correlated immunofluorescence and quantitative immunochemical study on serial cryosections. Eur Heart J, 1984. 5 Suppl F: p. 95-102.) detected both MYH6 and MYH7 in autoptic and bioptic specimens of human heart using specific anti-myosin antibodies. The authors report that MYH6 was less than 5% in most normal ventricular specimens and disappeared completely under the effect of pressure overload. On the other hand heavy chain beta was generally undetectable in the left atrial myocardium but increased up to 90% in biopsies of hypertrophied atria. Sato et al. (Sato, H., et al., [mRNA detection of beta-myosin heavy chain gene in the autopsy cases of hypertrophic cardiomyopathy]. Nippon Hoigaku Zasshi, 2000. 54(3): p. 408-13) also reports that overexpression of MYH7 correlates with sudden cardiac death suggesting that a dysregulated MYH expression contributes to pathological malfunction of the heart. (Garcia-Castro, M., et al., Hypertrophic cardiomyopathy: low frequency of mutations in the beta-myosin heavy chain (MYH7) and cardiac troponin T (TNNT2) genes among Spanish patients. Clin Chem, 2003. 49(8): p. 1279-85; Perrot, A., et al., Prevalence of cardiac beta-myosin heavy chain gene mutations in patients with hypertrophic cardiomyopathy. J Mol Med, 2005. 83(6): p. 468-77).

The identification of a novel microRNA in the MYH7 intron establishes that dysregulated expression of this miRNA may in fact be responsible for the cardiac disorders attributed to MYH7 itself. The specification provides compositions and methods relating to this discovery.

All publications, patents and patent applications mentioned in this specification are indicative of the level of skill of those skilled in the art to which this invention pertains. All publications, patents and patent applications cited herein are hereby incorporated by reference in their entirety.

1. An isolated nucleic acid molecule of less than 500 nucleotides characterized in that said isolated nucleic acid molecules comprises mir-208-2 (SEQ ID NO:7).

2. The isolated nucleic acid molecule of claim 1 wherein the length of said isolated nucleic acid molecule is less than 200 nucleotides.

3. The isolated nucleic acid molecule of claim 1 wherein the length of said isolated nucleic acid molecule is less than 100 nucleotides.

4. The isolated nucleic acid molecule of claim 1 wherein said isolated nucleic acid molecule is selected from the group consisting of SEQ ID NO:1, SEQ ID NO:2, SEQ ID NO:3 and SEQ ID NO:4.

5. The isolated nucleic acid molecule claim 1 wherein said isolated nucleic acid molecule consists of SEQ ID NO: 7.

6. The isolated nucleic acid molecule of claim 1 wherein said isolated nucleic acid molecule of claim 1 consists of a nucleic acid sequence which is complementary to a nucleic acid sequence selected from the group consisting of SEQ ID NO:1, SEQ ID NO:2, SEQ ID NO:3 and SEQ ID NO:4.

7. An isolated nucleic acid according to claim 6 consisting of SEQ ID NO:8.

8. The isolated nucleic acid molecule of claim 1 having between 8 and 50 nucleotides in length and capable of hybridizing under physiological conditions, preferably within a cell, to an isolated nucleic acid molecule of SEQ ID NO:8 and inhibiting the function of mir-208-2 (SEQ ID NO:7).

9. The isolated nucleic acid molecule of claim 8 wherein said isolated nucleic acid molecule is selected from the group consisting of SEQ ID NO:10 to SEQ ID NO:77.

10. A nucleic acid of claim 8 that is an antisense oligodeoxynudeotide (ASO), or a double stranded oligoribonucleotde (dsRNA), optionally comprising one or more chemical modifications selected from among:
a) a 3′ cap;
b) a 5′ cap,
c) a modified internucleoside linkage; or
d) a modified sugar or base moiety.

11. A nucleic acid of claim 1 comprising one or more chemical modifications selected from among:
a) a 3′ cap;
b) a 5′ cap,
c) a modified internucleoside linkage; or
d) a modified sugar or base moiety.

12. A nucleic acid vector comprising a nucleic acid sequence selected from the group consisting of SEQ ID NO:7 and SEQ ID NO:8, and at least one vector propagation sequence.

13. An isolated nucleic acid molecule according to claim 1 for use as a medicament.

14. A composition comprising a nucleic acid molecule according to claim 1 in a lipid or polymer based therapeutic delivery system.

15. A nucleic acid vector according to claim 10 for use as a medicament.

16. A cell comprising the nucleic acid vector of claim 12.

17. A method of treating a muscular disorder comprising administering to a subject having a muscular disorder a composition comprising an isolated nucleic acid molecule according to claim 13.

18. A method of treating a cardiovascular disorder comprising administering to a subject having a cardiovascular disorder a composition comprising an isolated nucleic acid molecule according to claim 13.

19. Use of a an isolated nucleic acid molecule according to claim 13 for the preparation of a medicament for treating a muscular disorder or a cardiovascular disorder.

20. A kit for use in diagnosing or determining a treatment strategy for a cardiovascular disorder comprising a nucleic acid reagent comprising a nucleic acid molecule according to claim 1, in either RNA, DNA, mixed RNA or DNA, and optionally any chemical modifications.

21. A method of reducing or increasing expression of mir-208-2 in a cell comprising administering to a cell a composition comprising an isolated nucleic acid molecule according to claim 1.

22. (Currently. Amended) Use of an isolated nucleic acid molecule of claim 13 characterized in that said isolated nucleic acid molecule comprises mir-208 (SEQ ID NO:6), and/or an isolated nucleic acid molecule of less than 500 nucleotides characterized in that said isolated nucleic acid molecules, comprise mir-499 (SEQ ID NO:9), and/or an isolated nucleic acid molecule of less than 500 nucleotides comprising the complementary sequence of mir-208 (SEQ ID NO:6) or of mir-499 (SEQ ID NO:9) for the preparation of a medicament for treating a muscular disorder or a cardiovascular disorder.

23. Use of an isolated nucleic acid molecule of claim 13 characterized in that said isolated nucleic acid molecule comprises mir-208 (SEQ ID NO:6), and/or an isolated nucleic acid molecule of less than 500 nucleotides characterized in that said isolated nucleic acid molecules comprise mir-499 (SEQ ID NO:9), and/or an isolated nucleic acid molecule of less than 500 nucleotides comprising the complementary sequence of mir-208 (SEQ ID NO:6) or of mir-499 (SEQ ID NO:9) for diagnosing a muscular disorder or a cardiovascular disorder.

24. A method of treating a patient comprising the use of a medicament according to claim 14.


SEQUENCE LISTING (77 sequences)

SEQ ID NO: 1 - DNA, 88 bp, homo sapiens
ccccacctccttctcctctcagggaagctttttgctcgaattatgtttctgatccgaatataagacgaacaaaaggtttgtctgaggg

SEQ ID NO: 2 - DNA, 88 bp, Pan troglodyte; identical to SEQ ID NO: 1

SEQ ID NO: 3 - DNA, 88 bp, canis familiaris
ccccagctccttctcctctcagggaagctttttgctcgcgttatgtttctcatccgaatataagacgaacaaaaggtttgtctgaggg

SEQ ID NO: 4 - DNA, 88 bp, rattus norvegicus
ccccacctcctgctcctctcagggaagctttttgctcgcgttatgtttctcatccgaatataagacgaacaaaaggtttgtctgaggg

SEQ ID NO: 5 - DNA, 22 bp, danio rerio
gtaagacgaacaaaaagttttt

SEQ ID NO: 6 - DNA, 22 bp, homo sapiens
ataagacgagcaaaaagcttgt

SEQ ID NO: 7 - DNA, 22 bp, homo sapiens
ataagacgaacaaaaggtttgt

SEQ ID NO: 8 - DNA, 22 bp, Homo sapiens
acaaaccttttgttcgtcttat

SEQ ID NO: 9 - DNA, 23 bp, homo sapiens
ttaagacttgcagtgatgtttaa

SEQ ID NO: 10 - DNA, 21 bp, homo sapiens
ccctcagacaaaccttttgtt

SEQ ID NO: 11 - DNA, 21 bp, Homo sapiens
cctcagacaaaccttttgttc

SEQ ID NO: 12 - DNA, 21 bp, Homo sapiens
ctcagacaaaccttttgttcg

SEQ ID NO: 13 - DNA, 21 bp, Homo sapiens
tcagacaaaccttttgttcgt

SEQ ID NO: 14 - DNA, 21 bp, Homo sapiens
cagacaaaccttttgttcgtc

SEQ ID NO: 15 - DNA, 21 bp, Homo sapiens
agacaaaccttttgttcgtct

SEQ ID NO: 16 - DNA, 21 bp, Homo sapiens
gacaaaccttttgttcgtctt

SEQ ID NO: 17 - DNA, 21 bp, Homo sapiens
acaaaccttttgttcgtctta

SEQ ID NO: 18 - DNA, 21 bp, Homo sapiens
caaaccttttgttcgtcttat

SEQ ID NO: 19 - DNA, 21 bp, Homo sapiens
aaaccttttgttcgtcttata

SEQ ID NO: 20 - DNA, 21 bp, Homo sapiens
aaccttttgttcgtcttatat

SEQ ID NO: 21 - DNA, 21 bp, Homo sapiens
accttttgttcgtcttatatt

SEQ ID NO: 22 - DNA, 21 bp, Homo sapiens
ccttttgttcgtcttatattc

SEQ ID NO: 23 - DNA, 21 bp, Homo sapiens
cttttgttcgtcttatattcg

SEQ ID NO: 24 - DNA, 21 bp, Homo sapiens
ttttgttcgtcttatattcgg

SEQ ID NO: 25 - DNA, 21 bp, Homo sapiens
tttgttcgtcttatattcgga

SEQ ID NO: 26 - DNA, 21 bp, Homo sapiens
ttgttcgtcttatattcggat

SEQ ID NO: 27 - DNA, 21 bp, Homo sapiens
tgttcgtcttatattcggatc

SEQ ID NO: 28 - DNA, 21 bp, Homo sapiens
gttcgtcttatattcggatca

SEQ ID NO: 29 - DNA, 21 bp, Homo sapiens
ttcgtcttatattcggatcag

SEQ ID NO: 30 - DNA, 21 bp, Homo sapiens
tcgtcttatattcggatcaga

SEQ ID NO: 31 - DNA, 21 bp, Homo sapiens
cgtcttatattcggatcagaa

SEQ ID NO: 32 - DNA, 21 bp, Homo sapiens
gtcttatattcggatcagaaa

SEQ ID NO: 33 - DNA, 21 bp, Homo sapiens
tcttatattcggatcagaaac

SEQ ID NO: 34 - DNA, 21 bp, Homo sapiens
cttatattcggatcagaaaca

SEQ ID NO: 35 - DNA, 21 bp, Homo sapiens
ttatattcggatcagaaacat

SEQ ID NO: 36 - DNA, 21 bp, Homo sapiens
tatattcggatcagaaacata

SEQ ID NO: 37 - DNA, 21 bp, Homo sapiens
atattcggatcagaaacataa

SEQ ID NO: 38 - DNA, 21 bp, Homo sapiens
tattcggatcagaaacataat

SEQ ID NO: 39 - DNA, 21 bp, Homo sapiens
attcggatcagaaacataatt

SEQ ID NO: 40 - DNA, 21 bp, Homo sapiens
ttcggatcagaaacataattc

SEQ ID NO: 41 - DNA, 21 bp, Homo sapiens
tcggatcagaaacataattcg

SEQ ID NO: 42 - DNA, 21 bp, Homo sapiens
cggatcagaaacataattcga

SEQ ID NO: 43 - DNA, 21 bp, Homo sapiens
ggatcagaaacataattcgag

SEQ ID NO: 44 - DNA, 21 bp, Homo sapiens
gatcagaaacataattcgagc

SEQ ID NO: 45 - DNA, 21 bp, Homo sapiens
atcagaaacataattcgagca

SEQ ID NO: 46 - DNA, 21 bp, Homo sapiens
tcagaaacataattcgagcaa

SEQ ID NO: 47 - DNA, 21 bp, homo sapiens
cagaaacataattcgagcaaa

SEQ ID NO: 48 - DNA, 21 bp, Homo sapiens
agaaacataattcgagcaaaa

SEQ ID NO: 49 - DNA, 21 bp, Homo sapiens
gaaacataattcgagcaaaaa

SEQ ID NO: 50 - DNA, 21 bp, Homo sapiens
aaacataattcgagcaaaaag

SEQ ID NO: 51 - DNA, 21 bp, Homo sapiens
aacataattcgagcaaaaagc

SEQ ID NO: 52 - DNA, 21 bp, Homo sapiens
acataattcgagcaaaaagct

SEQ ID NO: 53 - DNA, 21 bp, Homo sapiens
cataattcgagcaaaaagctt

SEQ ID NO: 54 - DNA, 21 bp, Homo sapiens
ataattcgagcaaaaagcttc

SEQ ID NO: 55 - DNA, 21 bp, Homo sapiens
taattcgagcaaaaagcttcc

SEQ ID NO: 56 - DNA, 21 bp, Homo sapiens
aattcgagcaaaaagcttccc

SEQ ID NO: 57 - DNA, 21 bp, Homo sapiens
attcgagcaaaaagcttccct

SEQ ID NO: 58 - DNA, 21 bp, Homo sapiens
ttcgagcaaaaagcttccctg

SEQ ID NO: 59 - DNA, 21 bp, Homo sapiens
tcgagcaaaaagcttccctga

SEQ ID NO: 60 - DNA, 21 bp, Homo sapiens
cgagcaaaaagcttccctgag

SEQ ID NO: 61 - DNA, 21 bp, Homo sapiens
gagcaaaaagcttccctgaga

SEQ ID NO: 62 - DNA, 21 bp, Homo sapiens
agcaaaaagcttccctgagag

SEQ ID NO: 63 - DNA, 21 bp, Homo sapiens
gcaaaaagcttccctgagagg

SEQ ID NO: 64 - DNA, 21 bp, Homo sapiens
caaaaagcttccctgagagga

SEQ ID NO: 65 - DNA, 21 bp, Homo sapiens
aaaaagcttccctgagaggag

SEQ ID NO: 66 - DNA, 21 bp, Homo sapiens
aaaagcttccctgagaggaga

SEQ ID NO: 67 - DNA, 21 bp, Homo sapiens
aaagcttccctgagaggagaa

SEQ ID NO: 68 - DNA, 21 bp, Homo sapiens
aagcttccctgagaggagaag

SEQ ID NO: 69 - DNA, 21 bp, Homo sapiens
agcttccctgagaggagaagg

SEQ ID NO: 70 - DNA, 21 bp, Homo sapiens
gcttccctgagaggagaagga

SEQ ID NO: 71 - DNA, 21 bp, Homo sapiens
cttccctgagaggagaaggag

SEQ ID NO: 72 - DNA, 21 bp, Homo sapiens
ttccctgagaggagaaggagg

SEQ ID NO: 73 - DNA, 21 bp, Homo sapiens
tccctgagaggagaaggaggt

SEQ ID NO: 74 - DNA, 21 bp, Homo sapiens
ccctgagaggagaaggaggtg

SEQ ID NO: 75 - DNA, 21 bp, Homo sapiens
cctgagaggagaaggaggtgg

SEQ ID NO: 76 - DNA, 21 bp, Homo sapiens
ctgagaggagaaggaggtggg

SEQ ID NO: 77 - DNA, 21 bp, Homo sapiens
tgagaggagaaggaggtgggg